(19) United States
(12) Patent Application Publication
(10) Pub. No.: US 2019/0008369 A1
(43) Pub. Date: Jan. 10, 2019

(54) ENDOSCOPE

(71) Applicant: OLYMPUS CORPORATION, Tokyo (JP)

(72) Inventors: Toshihiko HASHIGUCHI, Sagamihara-shi (JP); Kenichi NISHINA, Tokyo (JP); Teppei TSURUTA, Tokyo (JP); Yasuhiro IIJIMA, Tokyo (JP); Kei IRIE, Tokyo (JP)

(73) Assignee: OLYMPUS CORPORATION, Tokyo (JP)

(21) Appl. No.: 16/125,884

(22) Filed: Sep. 10, 2018

Related U.S. Application Data
(63) Continuation of application No. PCT/JP2016/086811, filed on Dec. 9, 2016.

(30) Foreign Application Priority Data
Mar. 14, 2016 (JP) 2016-049648

(51) Int. Cl. A61B 1/012; A61B 1/05; A61B 1/00

(52) U.S. Cl. CPC A61B 1/012; A61B 1/051; A61B 8/12; A61B 1/00078; A61B 1/00114; A61B 1/00018

(57) ABSTRACT

An endoscope includes: a rigid insertion portion inserted into a subject; an image sensor provided at a distal end of the insertion portion and configured to acquire an image of the subject; a rigid channel provided in the insertion portion, extended to be inclined with respect to a longitudinal axis of the insertion portion, and having a tubular shape into which a long member is inserted; and a signal cable including signal lines extended from the image sensor to transmit a signal acquired by the image sensor, and formed by winding a portion around an outer circumference of the channel at least one turn, wherein an arrangement of the channel and the signal cable at a first end side of the insertion portion is different from an arrangement of the channel and the signal cable at a second end side of the insertion portion.

### CROSS-REFERENCE TO RELATED APPLICATION

This application is a continuation of PCT International Application No. PCT/JP2016/086811 filed on Dec. 9, 2016, which claims the benefit of priority from Japanese Patent Application No. 2016-049648, filed on Mar. 14, 2016, the entire contents of which are incorporated herein by reference.

### BACKGROUND

The present disclosure relates to an endoscope.

In the related art, when organs and materials of a subject such as a patient are observed, a rigid endoscope or a flexible endoscope is used. For example, an operator such as a doctor performs an observation of an observation object based on information on characteristics of the observation object generated based on an ultrasonic echo received from an ultrasonic transducer by using an endoscope provided with the ultrasonic transducer that transmits and receives an ultrasonic wave at a distal end of an insertion portion.

The ultrasonic transducer includes a plurality of piezoelectric elements that convert an electric pulse signal into an ultrasonic pulse (an acoustic pulse) to irradiate it to the observation object and convert the ultrasonic echo reflected from the observation object into an electric echo signal to output it. Each piezoelectric element is electrically connected to an ultrasonic observation apparatus through a cable including a plurality of signal lines.

The insertion portion of the endoscope is provided with a treatment tool channel inserted into and passing through a treatment instrument or the like to extend from the distal end of the insertion portion. In the case of the rigid endoscope, the treatment instrument channel is a rigid tubular member and is provided from the distal end of the insertion portion to a proximal end side.

However, there is a case in which an arrangement of a rigid built-in element such as the treatment instrument channel is changed inside the insertion portion of the rigid endoscope. At this time, the treatment instrument and the cable interfere with each other, such that there is a case in which an outer diameter of the insertion portion is increased. As a technique for changing the arrangement of the built-in element and suppressing the increase in the diameter, there is known an ultrasonic endoscope provided with a guide passage that a signal cable is inserted and penetrates and guides the signal cable to extend to be inclined with respect to a longitudinal axis of the insertion portion, in a distal end configuration portion provided to a distal end of the insertion portion to hold an ultrasonic transducer (for example, see Japanese Patent Application Laid-Open No. 9-135833).

### SUMMARY

An endoscope according to one aspect of the present disclosure includes: a rigid insertion portion inserted into a subject; an image sensor provided at a distal end of the insertion portion and configured to acquire an image of the subject; a rigid channel provided in the insertion portion, extended to be inclined with respect to a longitudinal axis of the insertion portion, and having a tubular shape into which a long member is inserted; and a signal cable including signal lines extended from the image sensor to transmit a signal acquired by the image sensor, and formed by winding a portion around an outer circumference of the channel at least one turn, wherein an arrangement of the channel and the signal cable at a first end side of the insertion portion is different from an arrangement of the channel and the signal cable at a second end side of the insertion portion.

The above and other features, advantages and technical and industrial significance of this disclosure will be better understood by reading the following detailed description of the presently preferred embodiment of the disclosure, when considered in connection with the accompanying drawings.

### BRIEF DESCRIPTION OF THE DRAWINGS

FIG. 1 is a perspective view schematically illustrating a rigid endoscope system according to an embodiment;

FIG. 2 is a perspective view schematically illustrating a configuration of a case in which an optical visual tube is mounted in a rigid endoscope main body of the rigid endoscope system according to an embodiment;

FIG. 3 is a partial cross-sectional view schematically illustrating a configuration of a main portion of the rigid endoscope main body of the rigid endoscope system according to an embodiment;

FIG. 4 is a cross-sectional view schematically illustrating a configuration of a main portion of a rigid endoscope main body of a rigid endoscope system according to a first modification of an embodiment;

FIG. 5 is a cross-sectional view schematically illustrating a configuration of a main portion of a rigid endoscope main body of a rigid endoscope system according to a second modification of an embodiment;

FIG. 6 is a partial cross-sectional view schematically illustrating a configuration of a main portion of a rigid endoscope main body of a rigid endoscope system according to a third modification of an embodiment;

FIG. 7 is a partial cross-sectional view schematically illustrating a configuration of a main portion of a rigid endoscope main body of a rigid endoscope system according to a fourth modification of an embodiment;

FIG. 8 is a partial cross-sectional view schematically illustrating a configuration of a main portion of a rigid endoscope main body of a rigid endoscope system according to a fifth modification of an embodiment; and

FIG. 9 is a partial cross-sectional view schematically illustrating a configuration of a main portion of a rigid endoscope main body of a rigid endoscope system according to a sixth modification of an embodiment.

### DETAILED DESCRIPTION

Hereinafter, an embodiment for carrying out the present disclosure will be described with reference to the drawings. Further, the present disclosure is not limited to the embodiment described below. In addition, in the description of the drawings, the same parts are denoted by the same reference numerals.

### EMBODIMENT

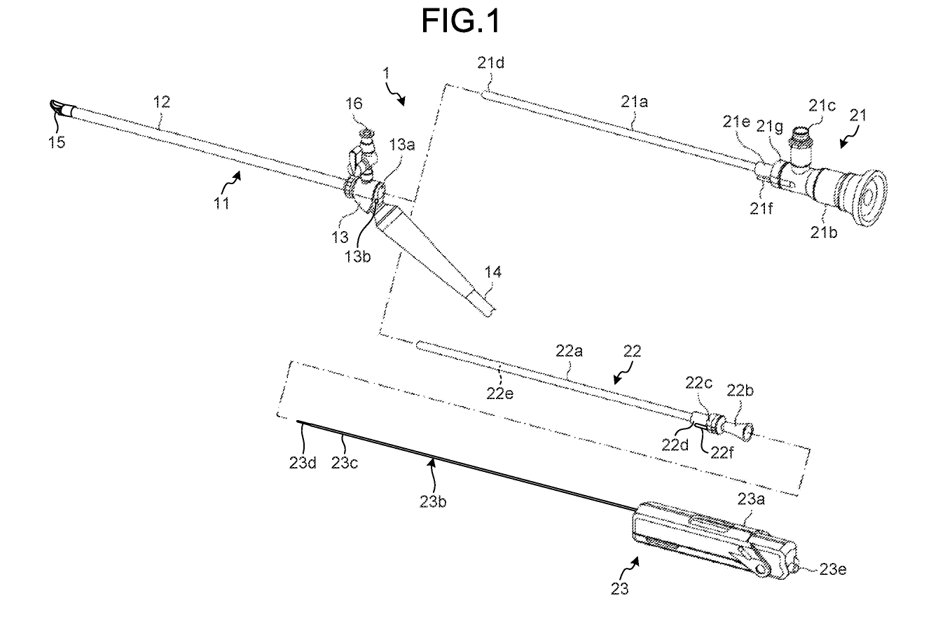

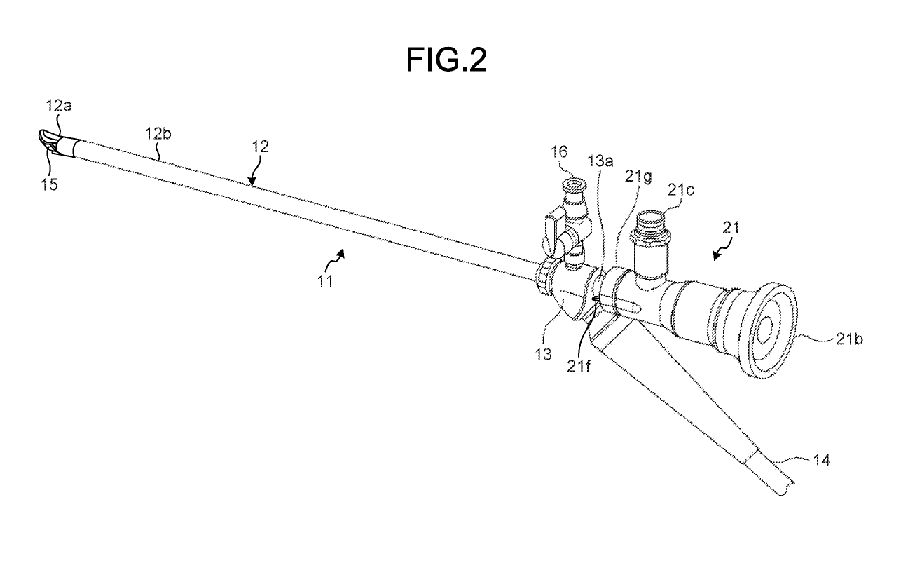

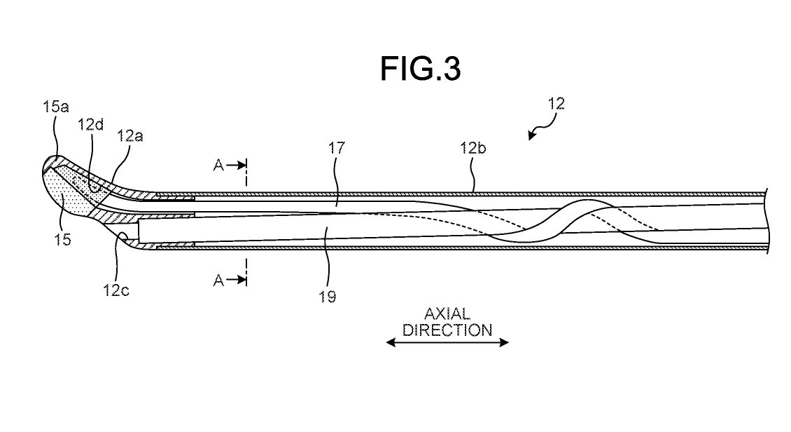

FIG. 1 is a perspective view schematically illustrating a rigid endoscope system according to an embodiment. FIG. 2 is a perspective view schematically illustrating a configuration of a case in which an optical visual tube is mounted in a rigid endoscope main body of the rigid endoscope system according to an embodiment. FIG. 3 is a partial cross-sectional view schematically illustrating a configuration of a main portion of the rigid endoscope main body of the rigid endoscope system according to an embodiment.

A rigid endoscope system 1 is a system performing ultrasonic diagnosis in a subject such as a human using an ultrasonic endoscope, and is used, for example, when a biological tissue of a prostate is transurethrally taken. The rigid endoscope system 1 includes a rigid endoscope main body 11, an optical visual tube 21 as an image device, a treatment instrument guide 22, and a treatment instrument device 23.

The rigid endoscope main body 11 includes a first insertion portion 12 inserted into a lumen (e.g., a urethra) of the subject, a grip portion 13 is provided to a hand side of the first insertion portion 12, and a universal cord 14 is extended from a side opposite to a side connected to the first insertion portion 12 of the grip portion 13. FIG. 2 illustrates a configuration of a case in which an optical visual tube 21 is mounted in the rigid endoscope main body 11, as an example of a use aspect of the rigid endoscope system 1.

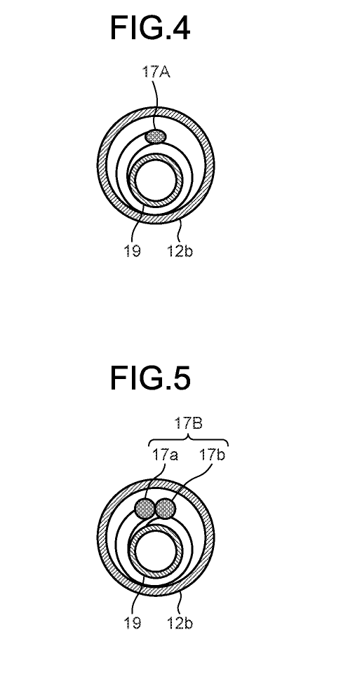

The first insertion portion 12 is rigid and is extended in a linear shape, and a signal cable 17 extending from the universal cord 14 is inserted into a lower side inside the first insertion portion 12 along an axial direction. The first insertion portion 12 includes a distal end configuring portion 12a provided to a distal end of the first insertion portion 12 to hold an ultrasonic transducer 15 for acquiring information of the subject, and a tubular portion 12b of a tubular shape formed by fitting a distal end into a proximal end side of the distal end configuring portion 12a and having a proximal end connected to the grip portion 13 (see FIGS. 3 and 4). In addition, in the distal end configuring portion 12a, a communication hole 12c that holds a first channel 19 described below and is in communication with the first channel 19, and a mounting portion 12d on which the ultrasonic transducer 15 is mounted are formed.

In addition, the ultrasonic transducer 15, which is an image sensor for acquiring the information on the subject is provided at the distal end of the first insertion portion 12. The ultrasonic transducer 15 is formed of, for example, an ultrasonic transducer of a convex array type, and is connected to a distal end portion of the signal cable 17. The ultrasonic transducer 15 includes a plurality of piezoelectric elements arranged along an axis core of the first insertion portion 12, and arranged so as to scan in a fan shape on an extension of the central axis of the first insertion portion 12. The ultrasonic transducer 15 converts an electrical pulse signal received from a controller, for example, a signal processing unit described below into an ultrasonic pulse (an acoustic pulse) to irradiate the ultrasonic pulse to the subject, and converts an ultrasonic echo reflected from the subject into an electrical echo signal represented in a voltage change to output it, by the piezoelectric elements provided at the distal end portion.

Further, the ultrasonic transducer 15 may be either of a convex vibrator and a linear vibrator. In the present embodiment, a description is made under the assumption that the ultrasonic transducer 15 is the ultrasonic transducer of the convex type in which the plurality of piezoelectric elements are provided in an array form and the piezoelectric elements associated with transmission and reception are electronically switched to electronically scan.

Further, although not illustrated, a connector is provided at a proximal end of the universal cord 14 and is connected to the signal processing unit. The signal processing unit transmits a driving signal to the ultrasonic transducer 15 through the signal cable 17, processes an echo signal generated according to an ultrasonic wave from the ultrasonic transducer 15, and generates an ultrasonic tomographic image to display it on a monitor (not illustrated).

In addition, a water supply port 16 with a cock is provided on an upper portion of the grip portion 13. The water supply port 16 is in communication with a first channel 19 described below and may supply a perfusion liquid through a perfusion tube (not illustrated). An operator can appropriately supply the perfusion liquid into the first channel 19 by opening the cock of the water supply port 16.

The first channel 19 is provided in the first insertion portion 12 so as to be inclined with respect to the axial direction of the first insertion portion 12. Specifically, a distal end portion of the first channel 19 is opened at a distal end surface of the first insertion portion 12 opposite to the grip portion 13 side and a proximal end portion of the first channel 19 is opened at a proximal end surface of the first insertion portion 12 of the grip portion 13 side. The proximal end portion of the first channel 19 is positioned at the water supply port 16 side in a diameter direction of the first insertion portion 12, and the distal end portion thereof is positioned at a side opposite to the water supply port 16 side in the diameter direction of the first insertion portion 12. The first channel 19 is, for example, a rigid tubular member formed by using stainless or the like. The first channel 19 preferably has a thickness of 0.15 mm to 0.20 mm in order to reduce an outer diameter of the first insertion portion 12. Further, in the present specification, a description is made under the assumption that a straight line passing through the respective centers of an opening of the distal end surface of the first insertion portion 12 opposite to the grip portion 13 side and an opening of the proximal end surface of the first insertion portion 12 of the grip portion 13 side is inclined with respect to a longitudinal axis of the tubular portion 12b.

In addition, in the grip portion 13, an insertion guide hole 13a of which a distal end is in communication with the first channel 19 and a proximal end is opened at the proximal end surface of the grip portion 13 is formed. Here, a positioning hole 13b is punched into the proximal end surface of the grip portion 13, such that a positioning pin protruding on the optical visual tube 21 and the treatment instrument guide 22 described below is inserted into the positioning hole 13b. Further, it is possible to retain the positioning pin by a fixing screw that fixes the positioning pin to the grip portion 13.

In addition, a second insertion portion 21a provided in the optical visual tube 21 and a third insertion portion 22a provided in the treatment instrument guide 22 are selectively inserted and removed into and from the first channel 19 of the rigid endoscope main body 11. Both the insertion portions 21a and 22a are rigid and are extended in a linear shape, and an inner diameter of the first channel 19 is set to a size suitable for an outer diameter of the second insertion portion 21a. Meanwhile, an outer diameter of the third insertion portion 22a is set to be approximately equal to the outer diameter of the second insertion portion 21a. In addition, a minute gap capable of allowing the perfusion liquid to flow is secured between an inner circumference of the first channel 19 and outer circumferences of both the insertion portions 21a and 22a. Therefore, the inner diameter of the first channel 19 is set to be slightly greater than the outer diameters of both the insertion portions 21a and 22a as much as the gap through which the perfusion liquid flows.

In addition, as illustrated in FIG. 1, an eyepiece portion 21b is provided to a hand side of the second insertion portion 21a provided in the optical visual tube 21, and a cap portion 21c into which a light guide (not illustrated) is inserted is provided at an upper portion in the vicinity of a distal end of the eyepiece portion 21b. The light guide extends in a distal end direction through the second insertion portion 21a, and illumination light transmitted through the light guide is radiated from an illumination window (not illustrated) provided at a distal end portion of the second insertion portion 21a and is irradiated to a body cavity of the subject. In addition, an observation window 21d is provided at the distal end of the second insertion portion 21a so as to be adjacent to the illumination window, and reflected light from the body cavity of the subject is incident to the observation window 21d and a subject image formed on an optical member such as an objective lens provided in the observation window 21d is transferred to the eyepiece portion 21b through a relay optical system and observed.

In addition, a flange portion 21g is formed at a distal end of the eyepiece portion 21b. A supporting portion 21e protrudes on the center of a distal end surface of the flange portion 21g. In addition, the supporting portion 21e supports the proximal end portion of the second insertion portion 21a. In a case in which the second insertion portion 21a is inserted into the rigid endoscope main body 11 through the insertion guide hole 13a, the distal end surface of the flange portion 21g faces the proximal end surface of the grip portion 13. In this case, the supporting portion 21e is inserted into and penetrates through the insertion guide hole 13a. In addition, a positioning pin 21f protrudes on a lower portion of the distal end surface of the flange portion 21g. The positioning pin 21f is inserted into the positioning hole 13b having the opening in the proximal end surface of the grip portion 13, such that a movement in a rotary direction is regulated.

The treatment instrument guide 22 has the third insertion portion 22a, an inducing portion 22b, a flange portion 22c, and a supporting portion 22d. The inducing portion 22b is provided at a hand side of the third insertion portion 22a and has a funnel shape. In addition, the flange portion 22c is formed at a distal end of the inducing portion 22b, and the supporting portion 22d protrudes on the center of a distal end surface of the flange portion 22c such that the proximal end of the third insertion portion 22a is supported by the supporting portion 22d. In a case in which the third insertion portion 22a is inserted into the rigid endoscope main body 11 through the insertion guide hole 13a, the distal end surface of the flange portion 22c faces the proximal end surface of the grip portion 13. In this case, the supporting portion 22d is inserted into and penetrates through the insertion guide hole 13a. In addition, a positioning pin 22f protrudes on a lower portion of the distal end surface of the flange portion 22c. The positioning pin 22f is inserted into the positioning hole 13b having the opening in the proximal end surface of the grip portion 13, such that a movement in a rotary direction is regulated.

In the third insertion portion 22a, a second channel 22e which is in communication with a guiding hole of which a distal end has an opening in the distal end surface of the third insertion portion 22a and a proximal end is formed at the inducing portion 22b is formed. An elongated rigid treatment instrument 23b extending forwardly in a linear shape from a device main body 23a provided in the treatment instrument device 23 may be inserted into and removed from the second channel 22e.

The second channel 22e serves as a guide when the treatment instrument 23b is inserted and removed, and an inner diameter of the second channel 22e is formed to be slightly greater than an outer diameter of the treatment instrument 23b. Further, in the present embodiment, the third insertion portion 22a is formed of a pipe material, a resin material is filled in the third insertion portion 22a, and the second channel 22e is formed in the filled resin material. Further, the third insertion portion 22a may form the second channel 22e by forming a hole in a solid metal material.

In the present embodiment, a biopsy device is illustrated as an example of the treatment instrument device 23, and a needle portion of the biopsy device corresponds to the treatment instrument 23b. Therefore, hereinafter, the treatment instrument device 23 is replaced with the biopsy device 23, or the treatment instrument 23b is replaced with the needle portion 23b.

The needle portion 23b has a guide cylinder needle 23c and a biopsy needle 23d having an outer diameter thinner than the second insertion portion 21a of the optical visual tube 21, and the biopsy needle 23d is inserted into and penetrates through the guide cylinder needle 23c so as to advance and retract. In addition, a pocket is formed at a distal end side of the biopsy needle 23d. The biopsy needle 23d receives repulsive force of a spring embedded in the device main body 23a by pushing a shooting button 23e provided on a rear surface of the device main body 23a and protrudes forwardly so as to be punched into a tissue of the subject, and a biopsy tissue is taken in the pocket. When the shooting button 23e is pushed, the guide cylinder needle 23c subsequent to the biopsy needle 23d protrudes, and when a distal end of the guide cylinder needle 23c passes over the pocket, the biopsy tissue is cut off and is taken into the pocket.

Since the first channel 19 is disposed at a position protruding on a scan surface (an observation view) of the ultrasonic transducer 15, the needle portion 23b passes through the scan surface of the ultrasonic transducer 15 when the needle portion 23b protrudes forwardly from the first channel 19, thereby making it possible to display the needle portion 23b on an ultrasonic tomographic image on a monitor.

The needle portion 23b according to the present embodiment is inserted into and penetrates through the third insertion portion 22a provided in the treatment instrument guide 22 with respect to the first channel 19. Therefore, in a case in which an outer diameter of the third insertion portion 22a is set to correspond to an inner diameter of the first channel 19 and an inner diameter of the second channel 22e formed in the third insertion portion 22a is set to correspond to an outer diameter of the needle portion 23b, the needle portion 23b which is thinner than the second insertion portion 21a of the optical visual tube 21 may accurately protrude on the scan surface of the ultrasonic transducer 15.

Next, an internal configuration of the rigid endoscope main body 11 will be described with reference to FIG. 3. In the signal cable 17, as illustrated in FIG. 3, a plurality of signal lines connected to a relay substrate 15a electrically connected to the ultrasonic transducer 15 and the signal cable 17, respectively, are gathered up in a bundle and extend to the grip portion 13 side. Further, an end portion opposite to a side connected to the relay substrate 15a of the signal cable 17 is connected to a connector (not illustrated) electrically connected to the universal cord 14 through the grip portion 13.

In the signal cable 17, a comprehensive shield is provided on an outer circumference formed by a signal line group including the plurality of signal lines described above, and a jacket, which is an outer skin, is provided on an outer circumference of the comprehensive shield. In addition, an end portion of a side that the signal cable 17 is connected to the relay substrate 15a, and an end portion of a side that the signal cable 17 is connected to the connector, that is, both end portions in a longitudinal direction are connected to the relay substrate 15a and the connector, respectively, in a state in which the comprehensive shield and the jacket are peeled off. Further, in the present embodiment, a cross section of the signal cable 17 perpendicular to a longitudinal axis is described as being circumscribed by a circle. In addition, an insulating pipe may be provided in an opening portion of a side which is inserted into and penetrates through the signal cable 17 of the mounting portion 12d. The comprehensive shield may be formed of a knitted strand (a braid) and may be formed of a strip-shaped tape.

As described above, since the first channel 19 is provided to be inclined with respect to the axial direction of the first insertion portion 12, the signal cable 17 interferes with the first channel 19 when the signal cable 17 is provided so as to extend in parallel to the central axis of the first insertion portion 12. For this reason, in the present embodiment, a position of the first channel 19 is fixed by the signal cable 17 by winding a portion of the signal cable 17 around the first channel 19, and an arrangement of the signal cable 17 and the first channel 19 is changed. Thus, the interference between the signal cable 17 and the first channel 19 can be avoided and the arrangement of the signal cable 17 and the first channel 19 can be changed.

Specifically, as illustrated in FIG. 3, the signal cable 17 and the first channel 19 are disposed to be in parallel to each other in a horizontal direction of the drawing at the distal end side. At such positions, the signal cable 17 is disposed at the distal end configuring portion 12a side and the first channel 19 is disposed at a side opposite to the distal end configuring portion 12a side.

The signal cable 17 is wound along the outer circumference of the first channel 19 when the signal cable 17 advances toward the grip portion 13 side along the longitudinal direction of the first insertion portion 12 from the above positional relationship. In this case, the first channel 19 slowly moves in a left direction along the inclination. After the signal cable 17 is wound along the outer circumference of the first channel 19, the arrangement of the signal cable 17 and the first channel 19 is changed to the arrangement opposite to the arrangement of the distal end configuring portion 12a side, that is, the arrangement rotated by 180°. As a result, by winding the signal cable 17 around the first channel 19, the diameter of the tubular portion 12b cannot be increased, the interference between the signal cable 17 and the first channel 19 can be avoided, and the arrangement of the signal cable 17 and the first channel 19 can be changed. In addition, in this case, the signal cable 17 is wound at least a half turn around the outer circumference of the first channel 19. Thus, the signal cable 17 has a function of a spacer between the first channel 19 and the tubular portion 12b and has a function of fixing the position of the first channel 19 in the wound portion.

According to the present embodiment described above, since the portion of the signal cable 17 having the plurality of signal lines that electrically connect between the ultrasonic transducer 15 and the connector is wound at least a half turn around the outer circumference of the first channel 19 having the rigid tubular shape extended to be inclined with respect to the longitudinal axis of the rigid first insertion portion 12, it is possible to suppress the increase in the diameter of the first insertion portion 12 and to change the arrangement of the signal cable and the channel.

In addition, according to the embodiment described above, since the signal cable 17 is wound at least a half turn around the outer circumference of the first channel 19, the signal cable 17 can achieve the function of the spacer between the first channel 19 and the tubular portion 12b and fix the position of the first channel 19 in the wound portion.

Further, although the embodiment described above describes that the arrangement of the signal cable and the channel is changed to the arrangement rotated by 180°, the arrangement of the signal cable and the channel is not limited thereto and the angle may be an angle other than 180°, for example, 90° or 45°, and if the spacer is present in the tubular portion 12b, the arrangement may not be changed. In a case in which the arrangement is not changed, when the signal cable 17 is wound at least a half turn around the outer circumference of the first channel 19, the signal cable 17 can achieve the function of the spacer between the first channel 19 and the tubular portion 12b and fix the position of the first channel 19 in the wound portion.

### First Modification of Embodiment

Although the embodiment described above describes that the cross section of the signal cable 17 perpendicular to the longitudinal axis is circumscribed by the circle, the cross section may be circumscribed by an ellipse and may be circumscribed by a long circular shape, a rectangular shape, or a polygonal shape. In the first modification, as an example, a configuration in which the cross section of the signal cable is circumscribed by the ellipse will be described. FIG. 4 is a cross-sectional view schematically illustrating a configuration of a main portion of a rigid endoscope main body of a rigid endoscope system according to a first modification of an embodiment, and illustrates a cross section corresponding to the line A-A of FIG. 3.

In a signal cable 17A according to the present first modification, as illustrated in FIG. 4, a shape formed by an outer edge, specifically, a shape formed by an outer circumference of an outer skin forms an ellipse. As in the present first modification, when the cross section of the signal cable 17A forms the ellipse, a short axial direction of the cross section of the signal cable 17A and a diameter direction of the first channel 19 are aligned with each other, and the signal cable 17A is wound around the first channel 19, an area occupied by the signal cable 17A and the first channel 19 in a diameter direction of the tubular portion 12b can be reduced.

According to the present first modification, the cross section of the signal cable 17A forms the ellipse, the short axial direction of the cross section of the signal cable 17A and the diameter direction of the first channel 19 are aligned with each other, and the signal cable 17A is wound around the first channel 19, thereby making it possible to achieve a decrease in a diameter of the tubular portion 12b as compared to the case using the signal cable 17 according to the embodiment described above.

### Second Modification of Embodiment

Although the embodiment described above describes that the signal cable 17 is arranged in a bundle, the signal cable 17 may be divided into a plurality of bundles. In the second modification, as an example, a configuration in which the plurality of signal lines of the signal cable are divided into two bundles will be described. FIG. 5 is a cross-sectional view schematically illustrating a configuration of a main portion of a rigid endoscope main body of a rigid endoscope system according to a second modification of an embodiment, and illustrates a cross section corresponding to the line A-A of FIG. 3.

As illustrated in FIG. 5, a signal cable 17B according to the second modification has a first composite cable 17a having one signal line group of signal line groups in which a plurality of signal lines connected to the relay substrate 15a are divided into two bundles, and a second composite cable 17b having the other signal line group. As in the present second modification, when the signal cable 17B is divided into two bundles and is arranged along the outer circumference of the first channel 19, an area occupied by the signal cable 17B and the first channel 19 in the diameter direction of the tubular portion 12b can be reduced.

According to the present second modification, the signal cable 17B is divided into the two bundles and is arranged along the outer circumference of the first channel 19, thereby making it possible to achieve a decrease in a diameter of the tubular portion 12b as compared to the case using the signal cable 17 according to the embodiment as described above.

### Third Modification of Embodiment

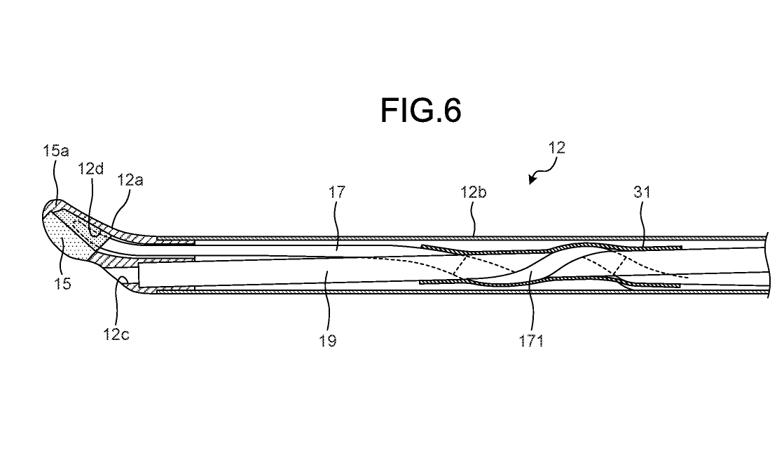

Although the embodiment described above describes that the signal cable 17 including the comprehensive shield and the jacket is wound around the first channel 19, thinness in a diameter may be achieved by removing the comprehensive shield and the jacket of the wound portion of the signal cable 17. FIG. 6 is a partial cross-sectional view schematically illustrating a configuration of a main portion of a rigid endoscope main body of a rigid endoscope system according to a third modification of an embodiment.

As illustrated in FIG. 6, the signal cable 17 according to the present third modification is formed by removing the comprehensive shield and the jacket of a portion wound around the first channel 19, and has an exposed portion 171 through which the plurality of signal lines are exposed. In the signal cable 17, the exposed portion 171 is wound around the first channel 19. In addition, a region including the exposed portion 171 of the signal cable 17 is coated with a heat shrinking tube 31. The heat shrinking tube 31 coats the exposed portion 171 and an arranged region of the exposed portion 171 in the first channel 19. Further, in the present third modification, a description is made that at least an outer surface of the first channel 19 has insulation properties by performing an insulation coating on the outer surface of the first channel 19 or winding an insulating tape such as a polyimide tape or the like around the outer surface of the first channel 19.

According to the present third modification, the exposed portion 171 formed by removing the comprehensive shield and the jacket of the portion wound around the first channel 19 and through which the plurality signal lines are exposed is formed, the exposed portion 171 is wound around the first channel 19, and the exposed portion 171 and the arranged region of the exposed portion 171 in the first channel 19 are coated with the heat shrinking tube 31. As a result, it is possible to prevent exposed portions (the exposed portion 171) of the signal lines of the signal cable 17 from being damaged and to achieve the decrease in the diameter of the tubular portion 12b as compared to the case using the signal cable 17 according to the embodiment described above.

### Fourth Modification of Embodiment

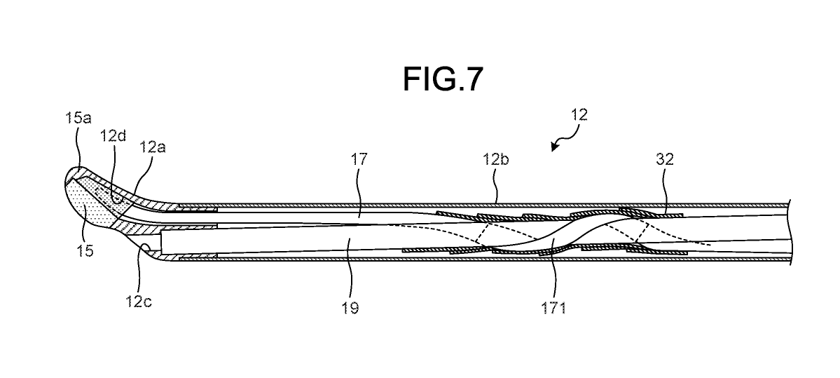

Although the third modification described above describes that the exposed portion 171 is formed by removing the comprehensive shield and the jacket of the wound portion of the signal cable and the exposed portion 171 is coated with the heat shrinking tube 31, insulation properties may be applied to the coated portion. FIG. 7 is a partial cross-sectional view schematically illustrating a configuration of a main portion of a rigid endoscope main body of a rigid endoscope system according to a fourth modification of an embodiment.

As illustrated in FIG. 7, the signal cable 17 according to the present fourth modification is formed by removing the comprehensive shield and the jacket of the portion wound around the first channel 19, and has the exposed portion 171 through which the plurality of signal lines are exposed, similarly to the third modification described above. In the signal cable 17, the exposed portion 171 is wound around the first channel 19. In addition, an insulating tape 32 is wound in a spiral shape around the region including the exposed portion 171 of the signal cable 17. As the insulating tape 32, an insulating member such as a polyimide tape or the like is used.

According to the present fourth modification, the exposed portion 171 formed by removing the comprehensive shield and the jacket of the portion wound around the first channel 19 and through which the plurality signal lines are exposed is formed, the exposed portion 171 is wound around the first channel 19, and the exposed portion 171 and the arranged region of the exposed portion 171 in the first channel 19 are coated with the insulating tape 32. As a result, it is possible to prevent exposed portions (the exposed portion 171) of the signal lines of the signal cable 17 from being damaged while securing insulation properties of the exposed portion 171, and to achieve the decrease in the diameter of the tubular portion 12b as compared to the case using the signal cable 17 according to the embodiment described above.

### Fifth Modification of Embodiment

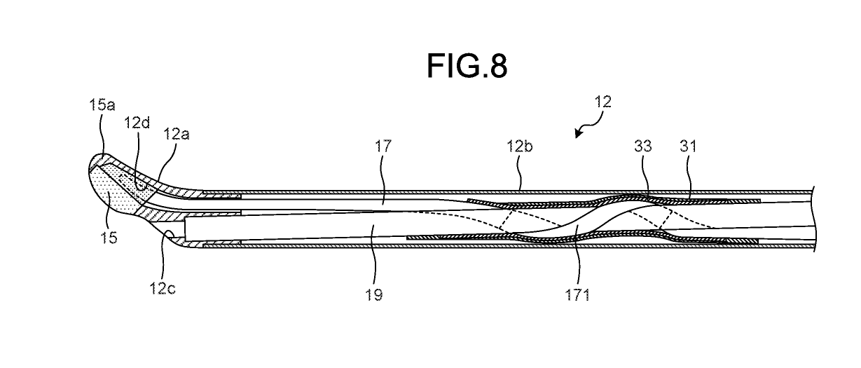

Although the third modification described above describes that the exposed portion 171 is formed by removing the comprehensive shield and the jacket of the wound portion of the signal cable and the exposed portion 171 is coated with the heat shrinking tube 31, noise resistance may be applied to the coated portion. FIG. 8 is a partial cross-sectional view schematically illustrating a configuration of a main portion of a rigid endoscope main body of a rigid endoscope system according to a fifth modification of an embodiment.

As illustrated in FIG. 8, the signal cable 17 according to the present fifth modification is formed by removing the comprehensive shield and the jacket of the portion wound around the first channel 19, and has the exposed portion 171 through which the plurality of signal lines are exposed, similarly to the third modification described above. The signal cable 17 winds the exposed portion 171 around the first channel 19. In addition, a region including the exposed portion 171 of the signal cable 17 is coated with a conductive tape 33 having conductivity, and the heat shrinking tube 31 is wound on an outer surface of the conductive tape 33. A portion of the conductive tape 33 is electrically connected to the comprehensive shield.

As the conductive tape 33, a metal foil having conductivity such as an aluminum tape or a copper tape, a braided tube (braid) formed using a conductive wire material, or a wire material used for the comprehensive shield is used. Further, if the metal foil has viscosity, it is preferable to improve workability in coating the heat shrinking tube, but for example, if the metal foil can be fixed to the heat shrinking tube at an end portion, the metal foil may be a metal foil that does not have viscosity.

According to the present fifth modification, the exposed portion 171 formed by removing the comprehensive shield and the jacket of the portion wound around the first channel 19 and through which the plurality signal lines are exposed is formed, the exposed portion 171 is wound around the first channel 19, the exposed portion 171 and the arranged region of the exposed portion 171 in the first channel 19 are coated with the conductive tape 33, and the conductive tape 33 is coated with the heat shrinking tube 31. As a result, it is possible to prevent exposed portions (the exposed portion 171) of the signal lines of the signal cable 17 from being damaged while securing noise resistance of a signal transmitted by the signal cable 17 and to achieve the decrease in the diameter of the tubular portion 12b as compared to the case using the signal cable 17 according to the embodiment described above.

Further, although the present fifth modification described above describes that the exposed portion 171 and the arranged region of the exposed portion 171 in the first channel 19 are coated with the heat shrinking tube 31, the exposed portion 171 may be coated with the insulating tape 32 instead of the heat shrinking tube 31, as in the fourth modification.

In addition, in the present fifth modification described above, at least an outer surface of the first channel 19 may have insulation properties by performing an insulation coating on the outer surface of the first channel 19, or winding the insulating tape such as the polyimide tape or the like on the outer surface of the first channel 19.

### Sixth Modification of Embodiment

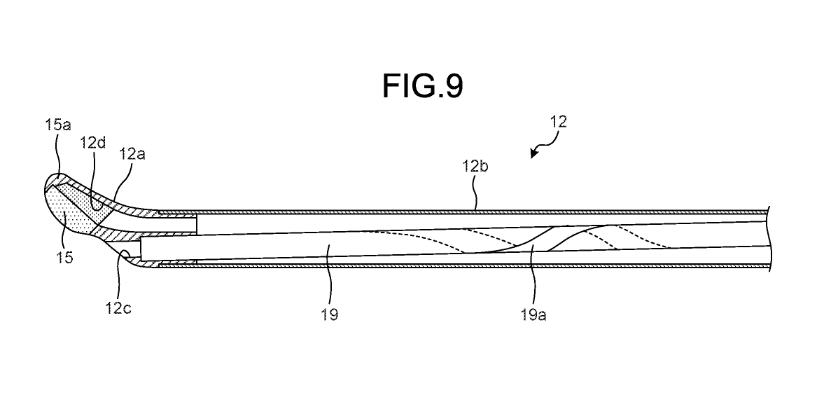

In the embodiment described above, the first channel 19 may be provided with a guiding portion for winding the signal cable 17. As an example, the present sixth modification describes a configuration in which the guiding portion has a groove extended in a concave shape formed on a surface of the first channel 19. FIG. 9 is a partial cross-sectional view schematically illustrating a configuration of a main portion of a rigid endoscope main body of a rigid endoscope system according to a sixth modification of an embodiment, and is a partial cross-sectional view illustrating a configuration except for the signal cable 17.

The first channel 19 according to the present sixth modification is provided with a guiding portion 19a for guiding a winding direction in which the signal cable 17 described above is wound, as illustrated in FIG. 9. The guiding portion 19a is formed by depressing the outer surface of the first channel 19, is formed in a concave shape, and is extended in a spiral shape along the outer surface of the first channel 19. Upon manufacturing the rigid endoscope main body 11, the signal cable 17 is wound along the depressed portion of the guiding portion 19a, thereby making it possible to regulate a winding position or a winding amount of the signal cable 17 for the first channel 19.

In addition, according to the present sixth modification, since the guiding portion 19a is formed by depressing the outer surface of the first channel 19 and is formed in the concave shape, a protruding amount of the signal cable 17 from the surface of the first channel 19 in the winding portion around the first channel 19 can be reduced as compared to the case that does not have the guiding portion 19a. Thereby, it is possible to reduce the outer diameter of the first insertion portion 12.

Further, although the present sixth modification describes that the guiding portion 19a forms the concave shape and is extended in the spiral shape along the outer surface of the first channel 19, the guiding portion 19a may form a convex shape and be extended in the spiral shape to thereby guide the signal cable 17, and may be a mark displaying (marking) the winding position of the signal cable 17 by ink or the like.

In addition, the guiding portion 19a may be extended along the winding direction of the signal cable 17, and may guide at least one of a winding start position, a winding position, and an intermittently indicated winding position.

Hereinabove, although the embodiment for carrying out the present disclosure has been described, the present disclosure is not limited to only the above-described embodiment and its modifications. The present disclosure is not limited to the above-described embodiment and its modifications, and can include various embodiments within the scope of the technical idea described in the claims. In addition, configurations of the embodiment and its modifications may be appropriately combined.

In addition, although the embodiment described above describe the piezoelectric element as an example of radiating the ultrasonic wave and converting the ultrasonic wave incident from the outside into the echo signal, the present disclosure is not limited to the piezoelectric element, and an element manufactured in a micro electro mechanical system (MEMS), for example, a capacitive micromachined ultrasonic transducers (C-MUTs) may be used.

In addition, the embodiment described above describe the ultrasonic endoscope observing the inside of the subject through the urethra as an example, the ultrasonic endoscope may be inserted into a biliary tract, bile duct, pancreatic duct, trachea, bronchial tube, or ureter to observe the surrounding organs (pancreas, lung, bladder, lymph nodes, etc.).

In addition, although the embodiment described above describe the ultrasonic endoscope as an example, the present disclosure is not limited thereto as long as it has the endoscope having the signal cable transmitting an image signal. For example, the present disclosure may be applied to an oral endoscope inserted into an alimentary tract (esophagus, stomach, duodenum, and large intestine), or respiratory organs (trachea and bronchial tubes) of the subject to perform an imaging of the alimentary tract or the respiratory organs, and an oral endoscope including a flexible insertion portion having an imaging element as image sensor. In particular, the present disclosure is useful in an endoscope including an image sensor having a large number of signal lines such as a charge coupled device (CCD) used in a high speed camera and having a cable requiring an insulating processing. According to the present disclosure, an effect capable of changing the arrangement of the signal cable and the channel while suppressing the increase in the diameter is achieved.

Additional advantages and modifications will readily occur to those skilled in the art. Therefore, the disclosure in its broader aspects is not limited to the specific details and representative embodiment shown and described herein. Accordingly, various modifications may be made without departing from the spirit or scope of the general inventive concept as defined by the appended claims and their equivalents.

What is claimed is:

1. An endoscope comprising:
a rigid insertion portion inserted into a subject;
an image sensor provided at a distal end of the insertion portion and configured to acquire an image of the subject;
a rigid channel provided in the insertion portion, extended to be inclined with respect to a longitudinal axis of the insertion portion, and having a tubular shape into which a long member is inserted; and
a signal cable including signal lines extended from the image sensor to transmit a signal acquired by the image sensor, and formed by winding a portion around an outer circumference of the channel at least one turn,
wherein an arrangement of the channel and the signal cable at a first end side of the insertion portion is different from an arrangement of the channel and the signal cable at a second end side of the insertion portion.

2. The endoscope according to claim 1, wherein
a cross section of the signal cable perpendicular to the longitudinal axis has an elliptical or long circular shape.

3. The endoscope according to claim 1, wherein
the signal cable includes signal line groups, each of the signal line groups including the signal lines.

4. The endoscope according to claim 1, wherein
the signal cable includes:
the signal lines;
an exposed portion exposing the signal lines in the signal cable; and
a comprehensive shield provided contiguously to the exposed portion and configured to coat the signal lines,
at least a surface of the channel has insulation properties,
the signal cable is wound around the channel in the exposed portion, and
the endoscope further comprises a coating tube configured to coat the signal cable including an outer circumference of the exposed portion, and the channel.

5. The endoscope according to claim 1, wherein
the signal cable includes:
the signal lines;
an exposed portion exposing the signal lines in the signal cable; and
a comprehensive shield provided contiguously to the exposed portion and configured to coat the signal lines,
at least a surface of the channel has insulation properties,
the signal cable is wound around the channel in the exposed portion, and
the endoscope further comprises an insulating tape wound in a spiral shape around the signal cable including an outer circumference of the exposed portion, and the channel.

6. The endoscope according to claim 1, wherein
the signal cable includes:
the signal lines;
an exposed portion exposing the signal lines in the signal cable; and
a comprehensive shield provided contiguously to the exposed portion and configured to coat the signal lines,
at least a surface of the channel has insulation properties,
the signal cable is wound around the channel in the exposed portion, and
the endoscope further comprises a conductive member configured to coat the signal cable including an outer circumference of the exposed portion, and the channel.

7. The endoscope according to claim 6, further comprising
a coating tube configured to coat an outer surface of the conductive member.

8. The endoscope according to claim 6, further comprising
an insulating tape wound in a spiral shape on an outer surface of the conductive member.

9. The endoscope according to claim 6, wherein
the conductive member is a conductive tape of a strip shape.

10. The endoscope according to claim 6, wherein
the conductive member is a braid formed of a conductive wire material.

11. The endoscope according to claim 4, wherein
the channel includes a conductive pipe member, and an insulation coating formed on an outer surface of the conductive pipe member.

12. The endoscope according to claim 4, wherein
the channel includes a conductive pipe member, and an insulating tape wound on an outer surface of the conductive pipe member.

13. The endoscope of claim 1, wherein
the channel has a guiding portion guiding a winding direction of the signal cable.

14. The endoscope according to claim 13, wherein
the guiding portion is provided on an outer surface of the channel and is extended in a concave shape.

15. The endoscope according to claim 13, wherein
the guiding portion is provided on an outer surface of the channel and is extended in a convex shape.

16. The endoscope according to claim 13, wherein
the guiding portion is a mark displayed on an outer surface of the channel.
